(19) United States
(12) Patent Application Publication
(10) Pub. No.: US 2010/0292759 A1
(43) Pub. Date: Nov. 18, 2010

(54) Magnetic field sensor for magnetically-coupled medical implant devices

(76) Inventors: Tae W. Hahn, Northridge, CA (US); Richard C. Ross, Westlake Village, CA (US)

Correspondence Address: Wong Cabello Lutsch Rutherford & Brucculeri LLP, 20333 Tomball Pkwy, Suite 600, Houston, TX, 77070, US

(21) Appl. No.: 11/089,171

(22) Filed: Mar. 24, 2005

(51) Int. Cl. A61F 11/04; A61N 1/375

(52) U.S. Cl. 607/57

(57) ABSTRACT

Disclosed is a cochlear stimulation system and associated methods that utilizes a magnetic field sensor to determine the status of magnetically-coupled components. The cochlear stimulation system includes an implantable portion positionable beneath the skin of a patient and an external portion positionable outside the skin of the patient. The implantable portion includes a multi-electrode array having a plurality of electrodes configured to be placed in cochlear duct of a patient and an internal magnet. The external portion includes a speech processor configured to generate control signals in response to received sound signals and an external magnet. The external magnet and the internal magnet generate an attractive magnetic force that maintains the external portion in position relative to the internal portion against the scalp of the patient. The cochlear stimulation system further includes a magnetic field sensor configured to sense the value of a magnetic field generated by the external magnet and the internal magnet in order to monitor changes in the magnetic field.

### TECHNICAL FIELD

This disclosure relates to systems and methods for stimulating the cochlea, and more particularly to systems and methods for detecting and measuring coupling status between magnetically-coupled components in a cochlear implant device.

### BACKGROUND

Prior to the past several decades, scientists generally believed that it was impossible to restore hearing to the deaf. However, scientists have had increasing success in restoring normal hearing to the deaf through electrical stimulation of the auditory nerve. The initial attempts to restore hearing were not very successful, as patients were unable to understand speech. However, as scientists developed different techniques for delivering electrical stimuli to the auditory nerve, the auditory sensations elicited by electrical stimulation gradually came closer to sounding more like normal speech. The electrical stimulation is implemented through a prosthetic device, called a cochlear implant, that includes an electrode array implanted in the inner ear to restore partial hearing to profoundly deaf people.

Such cochlear implants typically include an external portion that is not positioned under the skin, as well as an implanted portion that is positioned under the skin of the scalp. The external portion can include, for example, a headpiece or a behind-the-ear (BTE) unit that has a power source and a speech processor that processes incoming sound signals. The internal portion can include, for example, an implantable cochlear stimulator (ICS) and an electrode array. The ICS is implanted under the skin of the scalp behind the ear. The electrode array is inserted in a cochlear duct, usually the scala tympani, of the patient. One or more electrodes of the array selectively stimulate different auditory nerves at different places in the cochlea and at different times based on the pitch of a received sound signal.

The ICS and electrode array are implanted by cutting an incision in the skin of the scalp to form a skin flap behind the ear. A surgeon lifts the skin flap and inserts the ICS and the electrode array into the appropriate location relative to the patient's ear. The implanted portion and the external portion each include a magnet. The implanted portion is positioned relative to the external portion to result in a magnetic attraction between the two magnets. The magnetic attraction provides a holding force that maintains the external portion securely against the scalp and maintains the relative positions between the external portion and implanted portion. Proper positioning between the implanted portion and the external portion is necessary so that power and control signals can be optimally coupled from the external portion to the implanted portion.

### SUMMARY

The present inventors recognized that improved devices and methods were needed for monitoring and maintaining the status of the magnetic coupling between the external portion and the implanted portion of the cochlear implant. The disclosed devices and methods address this need.

In one aspect, a medical implant device includes an implantable portion positionable beneath skin of a patient, the implantable portion including an internal magnet; an external portion positionable outside the skin of the patient, the external portion including an external magnet, wherein the external magnet and the internal magnet generate an attractive magnetic force therebetween that maintains the external portion in position relative to the internal portion against skin of the patient; and a magnetic field sensor configured to output a value of a magnetic field generated by the external magnet and the internal magnet, wherein the sensor output can be used to obtain data indicative of the relative positioning of the internal portion and the external portion.

In another aspect, a method of monitoring the magnetically-coupled cochlear implant system includes deploying an implantable portion of a cochlear implant system under the skin of a patient; deploying an external portion of the cochlear implant over the skin of the patient; generating an attractive magnetic field between the implantable portion and the external portion to couple the implantable portion to the external portion; measuring a value of the magnetic field; and determining a baseline value of the magnetic field that indicates that the implantable portion is properly coupled to the external portion.

In another aspect, a cochlear stimulation system includes an implantable portion positionable beneath the skin of a patient and an external portion positionable outside the skin of the patient. The implantable portion includes an internal magnet and a multi-electrode array having a plurality of electrodes configured to be placed in cochlear duct of a patient. The external portion includes an external magnet and a speech processor configured to generate control signals in response to received sound signals. The external magnet and the internal magnet generate an attractive magnetic force that maintains the external portion in position relative to the internal portion against the scalp of the patient. The cochlear stimulation system further includes means for sensing the value of the magnetic field generated by the external magnet and the internal magnet in order to monitor changes in the magnetic field.

The details of one or more embodiments are set forth in the accompanying drawings and the description below. Other features and advantages will be apparent from the description and drawings, and from the claims.

### DESCRIPTION OF DRAWINGS

The features and advantages will be more apparent from the following more particular description thereof, presented in conjunction with the following drawings, wherein:

FIG. 1 shows a block diagram of an implanted system that uses the invention;

FIG. 2A illustrates a block diagram of an exemplary cochlear stimulation system that includes an implantable cochlear stimulator and an external headpiece connected with an external speech processor and power source;

FIG. 2B illustrates a block diagram of a behind-the-ear (BTE) cochlear stimulation system that includes an implanted cochlear stimulator and an external BTE unit that includes a power source, a speech processor and a microphone;

FIG. 3. shows a partial functional block diagram of a cochlear stimulation system, which system is capable of providing high rate pulsatile electrical stimuli and virtual electrodes;

FIG. 4 shows a schematic view of a Hall effect sensor in the absence of a magnetic field;

FIG. 5 shows a schematic view of a Hall effect sensor exposed to a magnetic field; and

FIG. 6 shows a flowchart that outlines one embodiment of a method of using a magnetic field sensor to monitor a coupling status between an external an implanted portion of a cochlear stimulation system.

Like reference symbols in the various drawings indicate like elements.

### DETAILED DESCRIPTION

Disclosed is an implanted system, such as a cochlear stimulation system, and associated methods that utilize a magnetic field sensor to monitor and determine the coupling status of magnetically-coupled components. It should be appreciated that the following description is exemplary and that the devices and methods described herein can be used with other types and other configurations of cochlear implant systems, as well as with other types of magnetically-coupled implant devices.

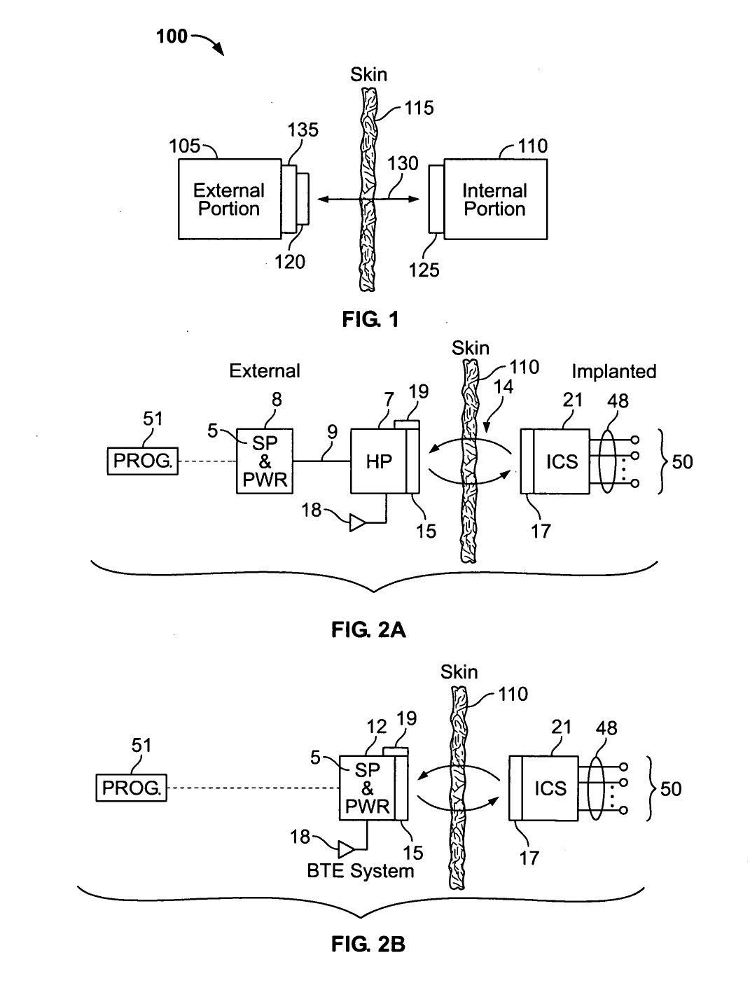

FIG. 1 shows an exemplary embodiment of an implanted system that incorporate the disclosed devices and methods. The implanted system 100 includes an external portion 105 and an internal portion 110. As used herein, the term “external” means not implanted under the skin or, in the context of an implanted cochlear system, not residing within the inner ear. However, the term “external” can also mean residing within the outer ear, residing within the ear canal or being located within the middle ear. The term “internal” or “implanted” means implanted under the skin.

With reference still to FIG. 1, the external portion 105 is positioned outside of the skin 115 and the internal portion 110 is positioned under the skin 115. An external magnet 120 is included on the external portion 105. An internal magnet 125 is included on the internal portion 110. The resulting magnetic field 130 between the two magnets 120, 125 provides a holding force that maintains the internal portion 110 and the external portion 105 in a fixed relationship relative to one another. The internal portion 105 and external portion 110 can also include respective coils that can be inductively or magnetically coupled.

A magnetic field sensor 135 is disposed on the external portion 105. The magnetic field sensor can also be disposed on the internal portion 110. The magnetic field sensor 135 is used to monitor and determine the coupling status of the magnetically-coupled external and internal portions of the implanted system 100. As described below, the magnetic field sensor 135 provides an output that is used to obtain data relating to the relative positioning of the internal portion 110 and the external portion 105. For example, the output of the magnetic field sensor 135 can be used to determine whether the external portion 105 has moved relative to the internal portion 110 or whether a magnetic lock between the two pieces has been compromised.

The implanted portion 110 and the external portion 105 can include additional components that enable functionality suited to the specific use of the implanted system 100. For example, FIGS. 2A and 2B show two embodiments of the implanted system used as cochlear stimulation systems (also referred to as cochlear implant systems), which typically include implanted and external components. The external components of the cochlear stimulation systems include a speech processor (SP) 5, a power source (e.g., a replaceable battery), and a headpiece (HP) 7. The SP 5 and power source are typically housed within a wearable unit 8 that is worn or carried by the patient, such as near the patient's waist. The wearable unit 8 is electrically connected to the headpiece 7, which is positioned adjacent the head, via a communication link, such as a cable 9. A microphone 18 is also included as part of the headpiece 7. The microphone 18 may be connected directly to the headpiece 7 or the SP 5 through an appropriate communication link.

In the embodiment shown in FIG. 2A, the implanted components include an implantable cochlear stimulator (ICS) 21 and an electrode array 48 having one or more electrodes 50. The ICS 21 is implanted behind the ear, so as to reside near the scalp. The ICS 21 and electrode array 48 are implanted by cutting an incision in the skin 110 of the scalp to form a skin flap behind the ear. A surgeon lifts the skin flap and inserts the ICS 21 and the electrode array 48 into the appropriate location relative to the ear.

The electrode array 48 is configured for implantation within the cochlea of the patient, usually in the scala tympani. The electrode array 48 is communicatively connected to the ICS 21. The array 48 includes a plurality of electrodes 50, e.g., sixteen electrodes, spaced along the array length and which electrodes are selectively connected to the ICS 21. The electrode array 48 may be substantially as shown and described in U.S. Pat. Nos. 4,819,647 or 6,129,753, both patents incorporated herein by reference. Electronic circuitry within the ICS 21 allows a specified stimulation current to be applied to selected pairs or groups of the individual electrodes included within the electrode array 48 in accordance with a specified stimulation pattern defined by the SP 5.

Inside of the headpiece 7 is a coil that is used to inductively or magnetically couple a modulated AC carrier signal to a similar coil that is included within the ICS 21. Thus, a data link 14 between the ICS 21 and the headpiece 7 and/or SP 5 is a transcutaneous (through the skin) data link that allows power and control signals to be sent from the SP 5 to the ICS 21. In order to achieve efficient coupling, without suffering significant losses in the signal energy, it is important that the external coil within the headpiece 7 be properly aligned with the internal coil inside the ICS 21. To achieve proper alignment, a first magnet 15 is included within the headpiece 7. A second magnet 17 is included within the ICS 21. The resulting magnetic attraction between the two magnets 15, 17 not only aligns the coils, as desired, but also provides a holding force that maintains the headpiece 7 securely against the scalp or skin 110 of the patient.

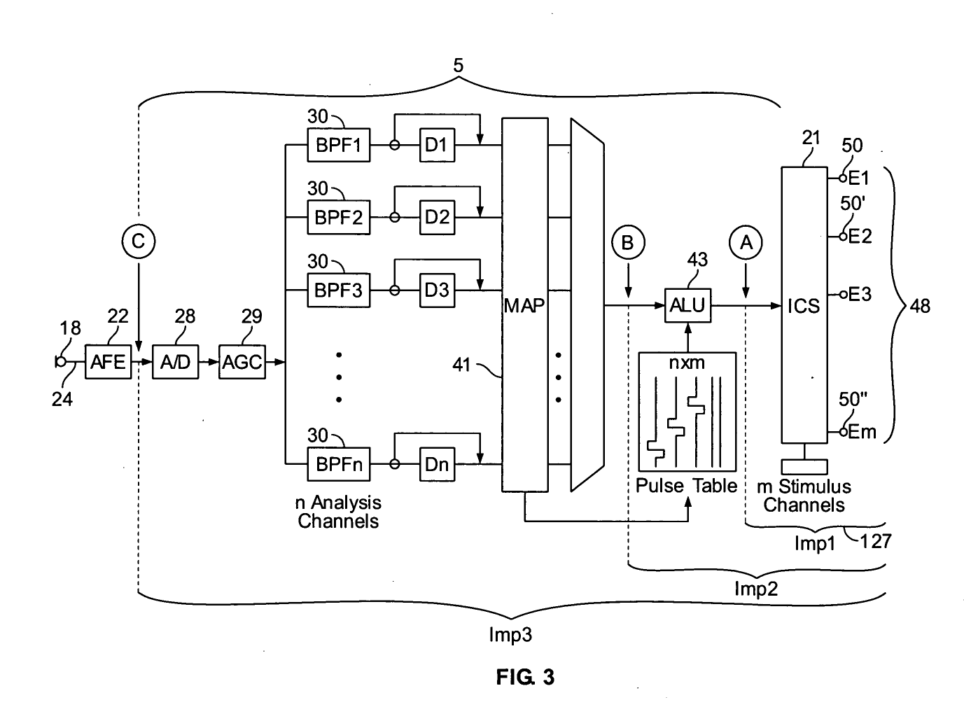

In use, a carrier signal is generated by circuitry within the wearable unit 8 using energy derived from the power source within the wearable unit 8. Such carrier signal, which is an AC signal, is conveyed over the cable 9 to the headpiece 7, where it is inductively coupled to the coil within the ICS 21. There it is rectified and filtered and provides a DC power source for operation of the circuitry within the ICS 21. Sounds are sensed through the external microphone 18 and amplified and processed by circuitry included within the speech processor unit 102. The sound signals are converted to appropriate stimulation signals in accordance with a selected speech processing strategy by the speech processor unit 102, as described further below with reference to FIG. 3. These stimulation signals modulate the carrier signal that transfers power to the ICS 21. The ICS 21 includes an appropriate demodulation circuit that recovers the stimulation signals from the modulated carrier and applies them to the electrodes 50 within the electrode array 48. The stimulation signals identify which electrodes, or electrode pairs, are to be stimulated, the sequence of stimulation and the intensity of the stimulation.

Some embodiments of the ICS 21 include a back telemetry feature that allows data signals to be transmitted from the ICS 21 to the headpiece 7, and hence to the SP 5. Such back telemetry data provides important feedback information to the speech processor regarding the operation of the ICS, including the amount of power needed by the ICS. Such back telemetry is described in U.S. Pat. No. 5,876,425, which is incorporated herein by reference.

When adjustment or fitting or other diagnostic routines need to be carried out on the system, an external programming unit 51 is detachably connected to the SP 5. Through use of the external programming unit 51, a clinician, or other medical personnel, is able to select the best speech processing strategy for the patient, as well as set other variables associated with the stimulation process. See, e.g., U.S. Pat. Nos. 5,626,629 or 6,289,247, incorporated herein by reference, for a more detailed description of a representative fitting/diagnostic process.

FIG. 2B shows another embodiment of the cochlear stimulation system 3. This embodiment incorporates a behind-the-ear (BTE) unit 12 that may include everything that was previously included within the wearable unit 8, only in a much smaller volume. The BTE unit 120 thus includes a suitable power source, as well as a speech processor 5 configured to perform a desired speech processing function. With the BTE unit 12, there is thus no need for the cable 9, and the patient simply wears the BTE unit behind his or her ear, where it is hardly noticed, especially if the patient has hair to cover the BTE unit.

The BTE unit 12 includes a magnet 15 that magnetically couples to a corresponding magnet 17 in the implanted ICS. As described above, the resulting magnetic attraction between the two magnets 15, 17 aligns the coils and also provides a holding force that maintains the BTE unit 12 securely against the scalp or skin 110 of the patient.

A pair of BTE units and corresponding implants can be communicatively linked via a Bionet and synchronized to enable bilateral speech information conveyed to the brain via both the right and left auditory nerve pathways. A system for allowing bilateral implant systems to be networked together is described in co-pending U.S. patent application Ser. No. 10/218,615, entitled “Bionet for Bilateral Cochlear Implant Systems”, which is incorporated herein by reference in its entirety and assigned to the same assignee as the instant application. The Bionet system uses an adapter module that allows two BTE units to be synchronized both temporally and tonotopically in order to maximize a patient's listening experience.

FIG. 3 shows a partial block diagram of one embodiment of a cochlear implant system capable of providing a high pusatile stimulation pattern and virtual electrodes, which are described below. At least certain portions of the SP 5 can be included within the implantable portion of the overall cochlear implant system, while other portions of the SP 5 can remain in the external portion of the system. In general, at least the microphone 18 and associated analog front end (AFE) circuitry 22 can be part of the external portion of the system and at least the ICS 21 and electrode array 48 can be part of the implantable portion of the system, as shown and described above in FIGS. 2A and 2B.

As mentioned, where a transcutaneous data link must be established between the external portion and implantable portions of the system, such link is implemented by using an internal antenna coil within the implantable portion, and an external antenna coil within the external portion. In operation, the external antenna coil is aligned over the location where the internal antenna coil is implanted, allowing such coils to be inductively coupled to each other, thereby allowing data (e.g., the magnitude and polarity of a sensed acoustic signals) and power to be transmitted from the external portion to the implantable portion. Note, in other embodiments, both the SP 5 and the ICS 21 may be implanted within the patient, either in the same housing or in separate housings. If in the same housing, the link 14 may be implemented with a direct wire connection within such housing. If in separate housings, as described, e.g., in U.S. Pat. No. 6,067,474, incorporated herein by reference, the link 14 may be an inductive link using a coil or a wire loop coupled to the respective parts.

The microphone 18 senses sound waves and converts such sound waves to corresponding electrical signals and thus functions as an acoustic transducer. The electrical signals are sent to the SP 5 over a suitable electrical or other link 24. The SP 5 processes these converted acoustic signals in accordance with a selected speech processing strategy to generate appropriate control signals for controlling the ICS 21. Such control signals specify or define the polarity, magnitude, location (which electrode pair or electrode group receive the stimulation current), and timing (when the stimulation current is applied to the electrode pair) of the stimulation current that is generated by the ICS. Such control signals thus combine to produce a desired spatio-temporal pattern of electrical stimuli in accordance with a desired speech processing strategy.

A speech processing strategy is used, among other reasons, to condition the magnitude and polarity of the stimulation current applied to the implanted electrodes of the electrode array 48. Such speech processing strategy involves defining a pattern of stimulation waveforms that are to be applied to the electrodes as controlled electrical currents.

As discussed above with reference to FIGS. 2A and 2B, a first magnet 15 in the headpiece 7 or BTE unit 12 and a second magnet 17 in the ICS 21 are used to maintains the headpiece 7 securely against the scalp or skin 110 of the patient. As shown in FIGS. 2A and 2B, the cochlear stimulation system further includes a magnetic field sensor 19 that is disposed in either the external or the implanted portion of the system. For example, the magnetic field sensor 19 can be deployed in the headpiece 7 in the embodiment of FIG. 2A or in the BTE unit 12 in the embodiment of FIG. 2B. Alternately, the magnetic field sensor 19 can be deployed in the ICS 21. The magnetic field sensor 17 provides an output that is used to obtain data relating to the relative positioning of the internal portion and the external portion of the cochlear stimulation system. For example, the output of the magnetic field sensor can be used to determine whether the headpiece 7 or BTE unit 12 has moved relative to the ICS 21 or whether the magnetic lock between the two pieces has been compromised.

The magnetic field sensor is described herein in an exemplary context of being a Hall effect sensor, although it should be appreciated that other types of magnetic field sensors can be used. A Hall sensor uses a thin sheet of conductive material (referred to as a Hall element) that is positioned in a magnetic field. In the case of the cochlear stimulation system, the magnetic field is the magnetic field generated by the first magnet 15 in the headpiece 7 or BTE unit 12 and/or the second magnet 17 in the ICS 21. When a current is applied to the Hall element, a voltage is generated perpendicular to both the current and the magnetic field. The voltage is proportional to the value of the magnetic field and current.

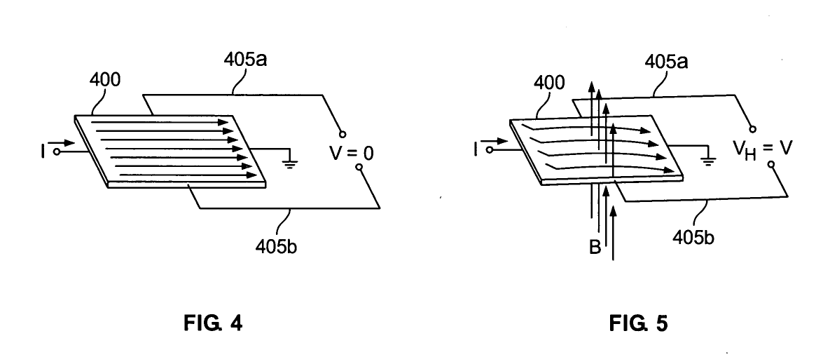

The foregoing process is referred to as the “Hall effect”, which is described in more detail with reference to FIGS. 4 and 5. FIG. 4 shows a Hall element 400, such as a thin piece of semiconducting material, through which a current I is passed. The Hall element 400 has a pair of output elements 405a, 405b that output from the Hall element perpendicular to the direction of the current I. When no magnetic field is present relative to the Hall element, the current distribution is uniform and no potential difference (i.e., no voltage) is seen across the output connections 405a, 405b.

With reference now to FIG. 5, a magnetic field B is shown present across the Hall element 400 along a direction perpendicular to the Hall element 400. Pursuant to the Hall effect, this results in a disturbance of the current distribution of the Hall element 400, thereby resulting in a potential difference (i.e., a voltage) across the output elements 405a, 405b. The voltage across the output elements 405a, 405b is referred to as the Hall voltage V_{H}. The Hall voltage V_{H} is proportional to the current I and the magnetic field B across the Hall element, as shown by the following equation:

V_{H}˜I×B

That is, the Hall voltage V_{H} is proportional to the vector cross product of the current I and the magnetic field B. The hall voltage V_{H} can be measured and stored using techniques and methods known in the art.

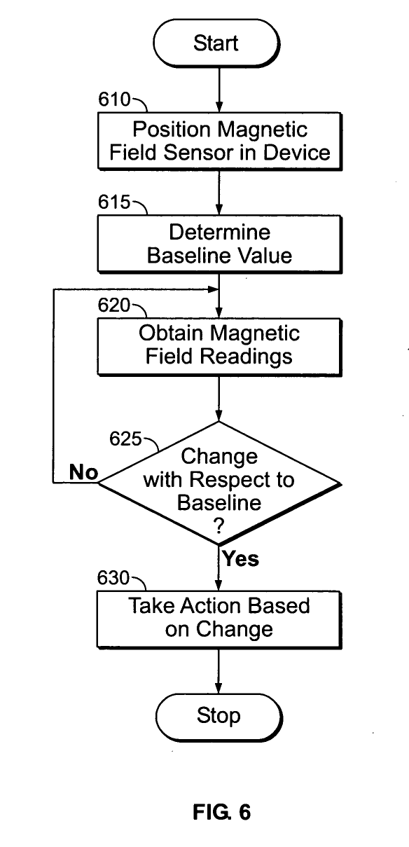

A method for using a magnetic field sensor in a cochlear stimulation system is now described. The method determines the coupling status between the external portion of the system and the implanted portion of the system. The process is described with reference to the flow diagram shown in FIG. 6. The flow diagram illustrates an exemplary method of using a magnetic field sensor in a cochlear stimulation system. Each step in the method shown in FIG. 6 is summarized in a block. The relationship between the steps i.e., the order in which the steps are carried out, is represented by the manner in which the blocks are connected in the flow chart. Each block has a reference number assigned to it.

In a first operation, represented by flow diagram box 610 in FIG. 6, the magnetic field sensor is deployed in the cochlear stimulation system. The magnetic field sensor can be deployed in the internal portion or the external portion of the system. For example, as shown in FIG. 2A, the magnetic field sensor 19 is deployed in the headpiece (HP). Alternately, as shown in FIG. 2B, the magnetic field sensor 19 is deployed in the BTE unit 12. In a scenario where the magnetic field sensor is a Hall sensor, a Hall element is deployed in the cochlear stimulation system such that the Hall element is positioned in a predetermined orientation relative to the magnetic field generated by the magnet 19 in the headpiece or the BTE unit. Specifically, the Hall element is oriented relative to the magnetic field such that a resulting Hall voltage is generated when a current is applied across the Hall element pursuant to the Hall effect described above. As discussed, the magnetic field sensor can alternately be attached to the implanted portion, such as to the ICS 21.

The magnetic field sensor is communicatively coupled to the speech processor 5, which is configured to calculate a metric associated with the value of the magnetic field measured by the magnetic field sensor 19. For example, the speech processor 5 can include a voltage meter that measures and outputs a value of the Hall voltage V_{H} in the scenario where the magnetic field sensor comprises a Hall sensor.

In the next operation, a baseline value of the magnetic field is determined, as represented by the flow diagram box 615. The baseline value is the measured magnetic field with the cochlear stimulation system in a predetermined state. For example, the baseline value can be the measured value of magnetic field when the magnet 15 in the external portion has achieved a sufficient lock with the magnet 17 in the internal portion. A sufficient lock is present where the external and internal portions are properly positioned with respect to one another such that the coils are properly aligned and a sufficient holding force maintains the headpiece 7 or BTE12 securely against the ICS 21 with the skin 110 of the patient interposed therebetween.

In the next operation, the magnetic field sensor continues to obtain readings of the measured magnetic field, as represented by the flow diagram box 620. The measured values are forward to the processor for analysis. The magnetic field is continuously measured, or measured on a regular interval, while the cochlear stimulation system is implanted and in use in the patient.

In the next operation, represented by decision box 625 in FIG. 6, it is determined whether the measured value of the magnetic field differs from the baseline value. This can be accomplished, for example, by employing a voltage comparator in the scenario where the magnetic field sensor comprises a Hall sensor. It should be appreciated that the measured value of magnetic field will differ from the baseline value if the magnet in the implanted portion has moved with respect to the magnet in the external portion of the cochlear stimulation system. This is because the magnetic field as measured at baseline is based on the predetermined relative positioning of the magnet 15 in the implanted portion and the magnet 17 in the external portion. The resultant magnetic field changes if the two magnets 15 and 17 move relative to one another. Thus, a change in the measured value of magnetic field with respect to the baseline can be an indication that the relative movement between the two magnets occurred and that the lock between the internal and external portion has been compromised or that the coils are no longer properly aligned.

If there is a change in the measured magnetic field with respect to baseline (a “Yes” output from decision box 625), then the process proceeds to the operation of flow diagram box 630, where appropriate corrective action is taken. For example, the headpiece 7, the BTE 12, or any other part of the system can be configured to emit an alarm, such as by generating a sound or causing a light to emit, that indicates that the lock between the internal and external portions of the implant has been compromised or terminated. Alternately, if the movement is small enough such that the magnet in the implanted portion is still locked to the magnet in the external portion, the processor may vary the power load to the implanted portion based on the movement between the two to ensure that the transcutaneous data link 14 between the ICS 21 and the headpiece 7 and/or SP 5 is maintained.

If it is determined that the measured magnetic field does not differ from the baseline value (a “No” output from the decision box 625), then the process returns to the operation of flow diagram box 620, where the magnetic field sensor continues to monitor the magnetic field. In this manner, the magnetic field sensor can be used to continually monitor the connection status between the external portion and the implanted portion of the cochlear stimulation system. Changes in such status are monitored by the magnetic field sensor.

A number of embodiments have been described. Nevertheless, it will be understood that various modifications may be made without departing from the spirit and scope of the claims. Accordingly, other embodiments are within the scope of the following claims.

1. A medical implant system comprising:
an implantable portion positionable beneath skin of a patient, the implantable portion comprising a first magnet and circuitry configured to control timing and amplitude of electrical stimulation delivered by the implantable portion;
an external portion positionable outside the skin of the patient, the external portion including a second magnet, wherein the second magnet and the first magnet generate a magnetic field therebetween to hold the external portion in position relative to the internal portion against skin of the patient; and
a magnetic field sensor associated with either the implantable portion or the external portion configured to output a value characterizing the magnetic field, wherein the sensor output can be used to infer the relative positioning of the internal portion and the external portion.

2. The system of claim 1, wherein the magnetic field sensor comprises a Hall effect sensor.

3. The system of claim 2, wherein the Hall sensor includes a Hall element oriented relative to the magnetic field such that a resulting Hall voltage is generated when a current is applied across the Hall element.

4. The system of claim 1, wherein the magnetic field sensor is configured to record a baseline value of the magnetic field, the baseline value corresponding to the second magnet being properly held to the first magnet.

5. The system of claim 4, further including a processor configured to compare a sensed value of the magnetic field to the baseline value of magnetic field.

6. The system of claim 1, wherein:
the medical implant system comprises a cochlear stimulation system;
the internal portion further includes a multi-electrode array having a plurality of electrodes configured to be placed in the cochlear duct of a patient; and
the external portion further includes a speech processor configured to generate control signals in response to received sounds.

7. The system of claim 6, wherein the external portion comprises a behind the ear (BTE) unit that includes the speech processor, the second magnet, and a power source, the BTE unit being positionable behind an ear of the user.

8. The system of claim 6, wherein the external portion comprises a wearable unit that includes the speech processor and a power source, the wearable unit being carried by the patient, the external portion further comprising a head unit positionable on the head of the user, the head unit including the speech processor and being coupled to the wearable unit by a cable.

9. The system of claim 6, wherein the system further comprises:
an acoustic transducer for sensing acoustic signals and converting them to electrical signals;
analog front end circuitry for preliminarily processing the electrical signals produced by the acoustic transducer; and
an implantable cochlear stimulator (ICS) connected to a the electrode array for generating electrical stimuli defined by the control signals from the speech processor.

10. The system of claim 9, wherein the analog front end circuitry and the acoustic transducer are included in the external portion.

11. The system of claim 1, wherein the magnetic sensor is included in the external portion.

12. A method of monitoring a magnetically-coupled cochlear implant system, comprising:
deploying an external portion of a cochlear implant over the skin of a patient above a deployed implantable portion of the cochlear implant system under the skin of the patient, wherein the deployed implantable portion includes circuitry configured to control timing and amplitude of electrical stimulation delivered by the implantable portion;
measuring a value of an attractive magnetic field generated between a magnet in the implantable portion and a magnet in the external portion, wherein the attractive magnetic field is of sufficient strength to hold the external portion to the implantable portion; and
determining a baseline value of the magnetic field to characterize the external portion as being properly held to the implantable portion.

13. The method of claim 12, further comprising:
determining that a measured value of the magnetic field differs from the baseline value; and
initiating corrective action to properly hold the external portion to the internal portion.

14. The method of claim 12, wherein measuring the value of the attractive magnetic field comprises:
applying a current through a Hall element positioned in the magnetic field;
measuring a resultant Hall voltage that is indicative of the value of the magnetic field.

15. The method of claim 12, wherein the implantable portion of the cochlear implant system under the skin of the patient comprises a multi-electrode array in the cochlea of the patient.

16. The method of claim 15, wherein the implantable portion of the cochlear system further comprises an implantable cochlear stimulator (ICS) connected to the multi-electrode array under the skin of the patient, the ICS configured to generate electrical stimuli defined by received control signals from a speech processor.

17. The method of claim 12, wherein the external portion of a cochlear system comprises a BTE unit adjacent an ear of the patient, the BTE unit comprising a speech processor, a power source, and a microphone.

18. A cochlear stimulation system comprising:
an implantable portion positionable beneath the skin of a patient, the implantable portion including
(i) a multi-electrode array having a plurality of electrodes configured to be placed in cochlear duct of patient,
(ii) a first magnet, and
(iii) circuitry configured to control timing and amplitude of electrical stimulation delivered by the implantable portion;
an external portion positionable outside the skin of the patient, the external portion including
(i) a speech processor configured to generate control signals in response to received sound signals, and
(ii) a second magnet, wherein the second magnet and the first magnet generate a magnetic field to hold the external portion in position relative to the internal portion against the scalp of the patient, and
(iii) a sensor for sensing a value of the magnetic field generated by the second magnet and the first magnet.

19. The cochlear stimulation system of claim 18, wherein the sensor comprises a Hall effect sensor.

20. The cochlear stimulation system of claim 18, wherein the sensor is attached to the external portion.

21. The cochlear stimulation system of claim 18, wherein the system further comprises:
an acoustic transducer for sensing acoustic signals and converting them to electrical signals;
analog front end circuitry for preliminarily processing the electrical signals produced by the acoustic transducer;
an implantable cochlear stimulator (ICS) connected to the electrode array for generating electrical stimuli defined by the control signals from the speech processor.
